(19) United States
(12) Patent Application Publication
(10) Pub. No.: US 2022/0125319 A1
(43) Pub. Date: Apr. 28, 2022

(54) Moisture and Fever Sensing Apparatus

(71) Applicant: Farnaz Zand, Burbank, CA (US)

(72) Inventor: Farnaz Zand, Burbank, CA (US)

(21) Appl. No.: 17/463,087

(22) Filed: Aug. 31, 2021

Related U.S. Application Data
(60) Provisional application No. 63/106,185, filed on Oct. 27, 2020.

(51) Int. Cl. A61B 5/01; G08B 21/02; G01K 11/16; G01K 13/20; A61B 5/00; A61F 13/42

(52) U.S. Cl. CPC A61B 5/01; A61F 2013/8473; G01K 11/16; G01K 13/20; A61B 5/002; A61B 5/0022; A61B 5/0008; A61B 5/7405; A61B 5/742; A61B 5/746; A61B 5/6808; A61F 13/42; A61B 2562/0276; A61F 2013/422; G08B 21/0211

(57) ABSTRACT

A combined apparatus for indicating the elevated temperature of an infant and for sensing moisture in a diaper. The combined apparatus includes an elongated sensing strip made of highly efficient liquid absorption and transfer capillary wick material having a first end disposed in engagement with the diaper. The elongated sensing strip has a first side having a pair of screen-printed, spaced apart conductive ink bands which couple electrically at one end with a portable alarm unit and a second side having an elongated band of thermochromic ink printed thereon a segment of which is visible proximate the second end of the elongated sensing strip.

### CROSS-REFERENCE TO RELATED APPLICATIONS

This is a Non-Provisional application claiming the benefit of Provisional Application No. 63/106,185 fled Oct. 27, 2020.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

The present invention relates generally to patient care devices and more particularly to a novel moisture sensing and elevated temperature indicating device. The combined apparatus of the invention continuously monitors the patient's temperature, wirelessly notifies caregiver of a dangerously high temperature and senses urination in clothing articles such as diapers worn by young children and/or incontinent adults, respectively.

### Description of Related Art Including Information Disclosed Under 37 CFR 1.97 and 1.98

Bladder incontinence is a problem endemic in the very young and is cured only by intensive training as the young child goes through its second and third year of life requiring in the meantime, the constant use of diapers. The same problem can affect the elderly or the infirm or persons of any age who may have this problem because of physiological or psychological problems. A problem of this nature may be a major source of embarrassment. The mere fear of such incontinence can be a danger to the psychological welfare of persons already subject to severe psychological pressures due to other infirmities. Such people, both the very young and the infirm, often have no warning that their undergarments are being wet by urine until the undergarments are uncomfortably wet. There has always been a need for a device which can automatically warn either the person involved, or his/her caretaker (nurse or parent) that such undesirable leakage or wetness has occurred.

Various devices for detecting moisture or wetness such as caused by urination are known in the prior art. In diapers, the purpose of such devices is to set off an alarm when the diaper becomes wet. This permits a parent or other attendant to immediately tend to a newborn infant, toddler or an incontinent adult. Such devices usually include a pair of electrodes placed in the diaper which conduct electric current if wetness is detected. Prior art devices of this type, however, have numerous disadvantages such as requiring current-carrying conductors to pass mechanically through the diaper's plastic outer sheath, which may subject the skin of the child/incontinent adult to potentially high voltages, may be sensitive only in a limited area in the diaper, may accidentally respond to the wearer sitting on a wet or metal bench or park slide or may have other undesirable drawbacks.

The numerous disadvantages of the prior art urine sensing devices have been largely overcome by the highly novel and useful apparatus illustrated and described in U.S. Pat. No. 6,559,772 issued to the inventor named herein. As will become apparent from the discussion which follows, the novel and useful apparatus described in this patent has now been improved by the addition of novel means for continuously monitoring the temperature of the patient while at the same time standing ready to automatically alert a caregiver that a diaper change is needed.

Similarly, a wide variety of temperature sensing devices that operate on several different scientific principles have been suggested in the past. By way of example, one type of prior art thermometer uses thermochromic liquid crystals that are constructed in a planar shape and react to changes in temperature by changing color. This type of thermometer is discussed in U.S. Pat. No. 6,241,386 issued to Limburg et al. As discussed in this patent, thermochromic liquid crystals are typically made of twisted molecular structures comprising optically active mixtures of organic chemicals and include cholesteric compositions, chiral nematic formulations, and combinations of the two. Such crystals have been implemented in a variety of forms. One of the most prevalent examples is a temperature strip made by placing numerous thermochromic liquid crystal rectangles end to end. The rectangles ae arranged so that, as the ambient temperature changes from lowest to highest readable temperature, the rectangles are individually illuminated sequentially from one end of the strip to the other.

Another prior art product that displays a change responsive to changes in temperature is discussed in U.S. Pat. No. 4,744,113 issued to Kogut. This product concerns a toilet training aid having a porous sheet of paper with an invisible picture thereon that is placed over a toilet bowl and produces an image when wetted by warm liquid.

In a similar vein, U.S. Pat. No. 4,725,462 issued to Kimura concerns the use of heat activated indicia on textiles. More particularly, Kimura uses thermochromic colors formed into a textile to form an image. The indicia is not visible to the naked eye in the normal ambient temperatures but when subjected to a predetermined temperature, such as when immersed in bath water, an image appears to the user.

### SUMMARY OF THE INVENTION

The present invention is directed to a combined apparatus for continuously monitoring the temperature of patients including infants and adults, wirelessly notifying the caregiver of a dangerously high temperature and at the same time functioning to sense moisture at the patient's diaper. The combined apparatus includes an elongated sensing strip made of highly efficient liquid absorption and transfer capillary wick material having a first end disposed in engagement with the diaper. The elongated sensing strip has a first side having a pair of screen-printed spaced apart conductive ink bands which couple electrically at one end with a portable alarm unit and a second side having an elongated band of thermochromic ink printed thereon a segment of which is visible proximate the second end of the elongated sensing strip. In one embodiment of the invention, a color sensor for sensing the change in color of the thermochromic ink is provided. The color sensor senses the change in color of the thermochromic ink indicating an increase in temperature and signals a receiving and transmitting means that is operably associated with the sensor. In turn, the receiving and transmitting means notifies the caregiver by wirelessly forwarding the signal to a selected one of multiple types of devices such as computers, speakers and cell phones.

In accordance with one aspect of the present invention, the sensing strip is made of a highly efficient capillary liquid transfer wick material.

In accordance with another aspect of the present invention, the sensing strip comprises a substantially short first portion disposed at the first location, a substantially long second portion disposed adjacent the substantially short first portion and a third portion disposed adjacent the second portion.

In accordance with still another aspect of the present invention, the sensing means includes at least two spaced apart conductive ink bands screen-printed on a first side of the second and third portions of the wick strip, a waterproof pouch for enclosing the second portion, and an alarm signaling circuit operatively coupled to the conductive ink bands on the third portion. The waterproof pouch comprises an opening at a first end and a pocket at a second end for removably receiving a portable alarm unit containing the alarm signaling circuit, the substantially short first portion of the wick strip protruding from the first end.

In accordance with yet another extremely important aspect of the present invention, the sensing apparatus of the present invention further includes an elongated band of thermochromic ink that is printed on the opposite side of the wick strip from the site upon which the conductive ink bands are printed. During use, a portion of this band is clearly visible to the caregiver proximate the second end of the elongated sensing strip.

These and other aspects of the present invention will become apparent from a review of the accompanying drawings and the following detailed description of the preferred embodiments of the present invention.

### BRIEF DESCRIPTION OF THE DRAWINGS

FIG. 1 is a side elevational view of a combined temperature indicating and moisture sensing apparatus in accordance with the present invention;

FIG. 2 is a perspective view of an undergarment (such as a diaper) with the temperature indicating and moisture sensing apparatus of FIG. 1 applied to the undergarment in accordance with the present invention;

FIG. 3 is a cross-sectional view taken along section line 3-3 of FIG. 2;

FIG. 4 is a front perspective view of a diaper insert partially enclosing a sensing strip in accordance with the present invention;

FIG. 5 is a back perspective view of a diaper insert partially enclosing a sensing strip in accordance with the present invention;

FIG. 6 is a top view of a sensing strip in accordance with the present invention;

FIG. 7 is a schematic view of the moisture sensor of FIG. 6 shown coupled to an electrical circuit in accordance with the present invention;

FIG. 8 is a bottom view of a sensing strip in accordance present invention;

FIG. 9 is a generally perspective, diagrammatic view of the sensing strip and the alarm signal unit of the present invention;

FIG. 10 is a fragmentary view similar to FIG. 3 looking into the undergarment at the body side of the pouch-like diaper insert and sensing strip of the invention;

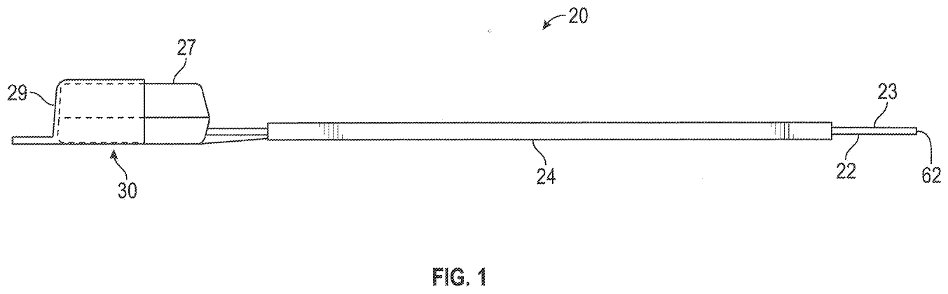

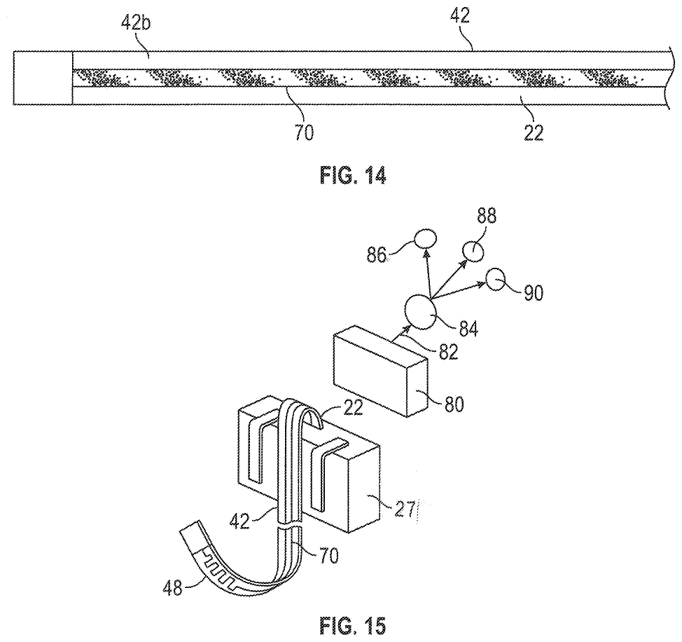

FIG. 1I is a greatly enlarged view of the area identified as 11-11 in FIG. 10;

FIG. 12 is a perspective view of an alternate form of the invention showing an undergarment with the temperature indicating and moisture sensing apparatus of FIG. 1 applied to the undergarment and showing a further form of temperature indicating apparatus involving the use of a color sensor operably associated with the sensing strip for sensing the change of color of a thermochromic ink applied to the strip;

FIG. 13 is a cross-sectional view taken along section line 13-13 of FIG. 12;

FIG. 14 is a bottom view of a sensing strip of this later form of the invention;

FIG. 15 is a generally perspective, fragmentary view of a sensing strip having thermochromic ink applied thereto, a color sensor for sensing the change in color of the thermochromic ink on the sensing strip indicating an increase in temperature and receiving and transmitting means operably associated with the sensor for notifying the caregiver of the increase in temperature.

### DESCRIPTION OF THE PREFERRED EMBODIMENT

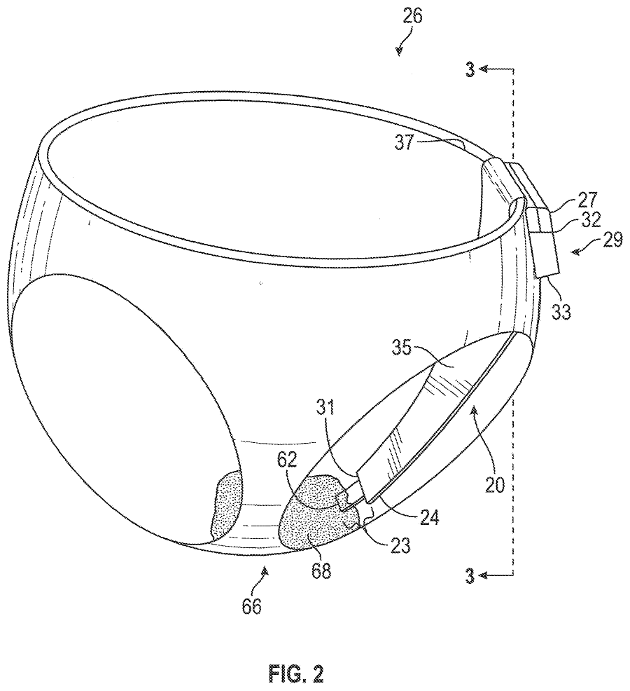

The present invention is directed to a combination elevated temperature sensing and moisture sensing apparatus generally designated in the drawings by the numeral 20. The apparatus continuously monitors temperature of the user and functions to detect urine in clothing articles such as diapers for young children and/or incontinent adults and here comprises a disposable pouch-like diaper insert 24 partially enclosing a disposable sensor in the form of a sensing strip 22. Diaper insert 24 is preferably designed to be waterproof on the inside, i.e. around the enclosed portion of strip 22, and is adapted on one side to be adhesively affixed to the inside of a young child's or an incontinent adult's diaper 26 (FIGS. 2, 3 and 5).

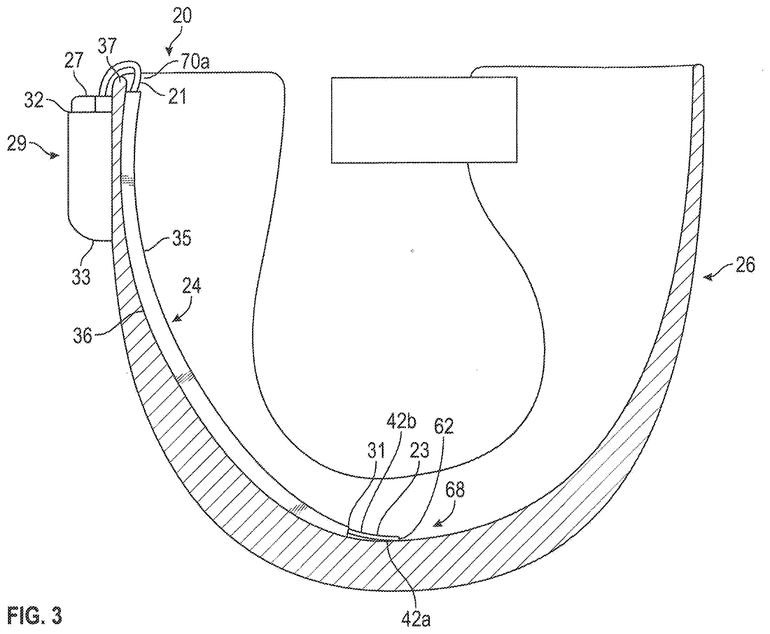

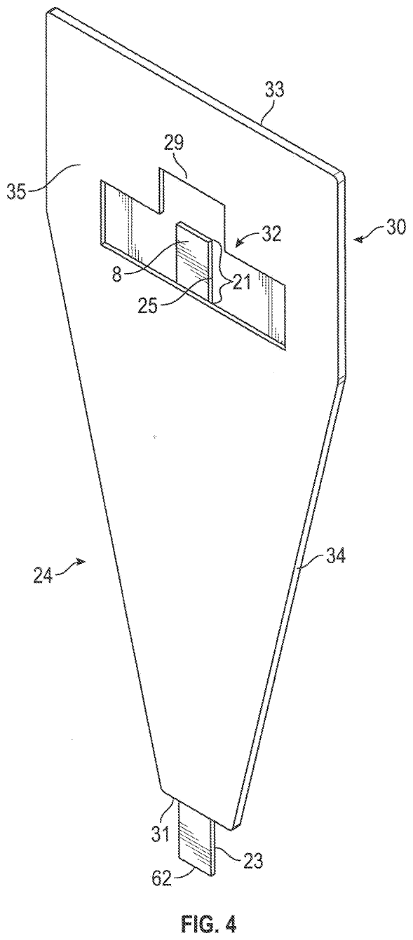

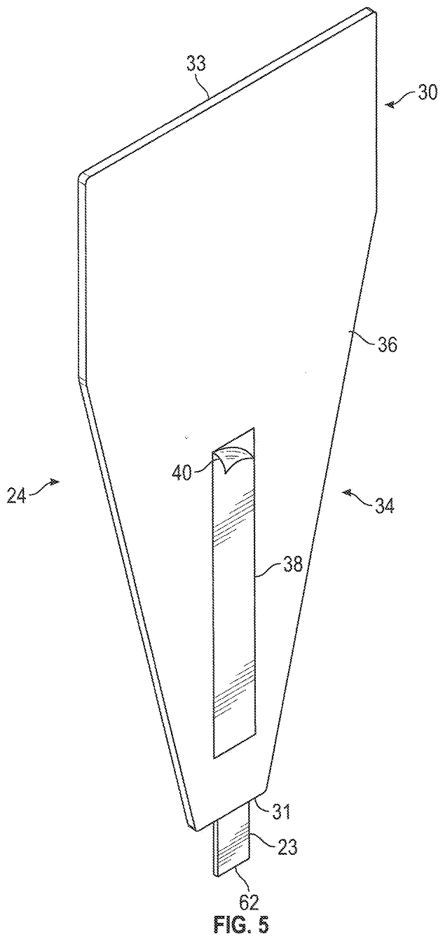

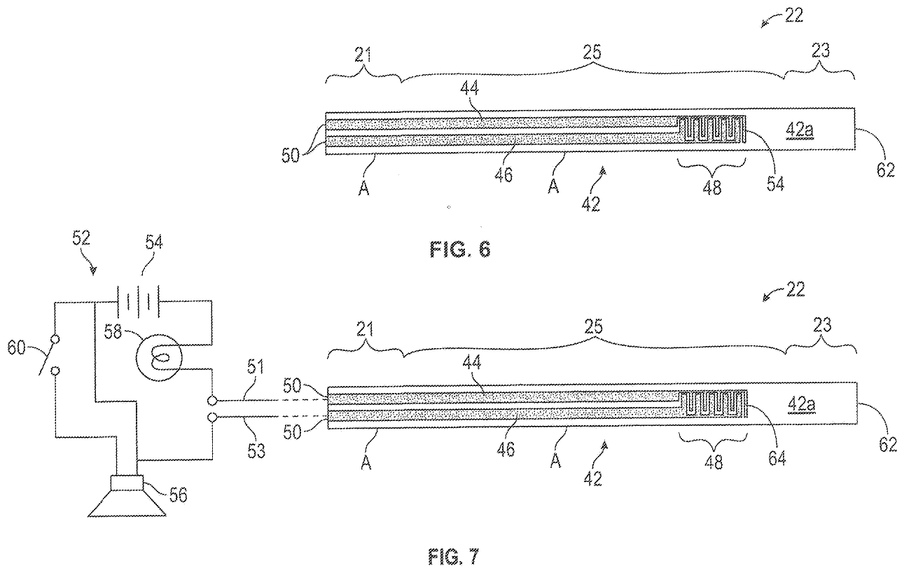

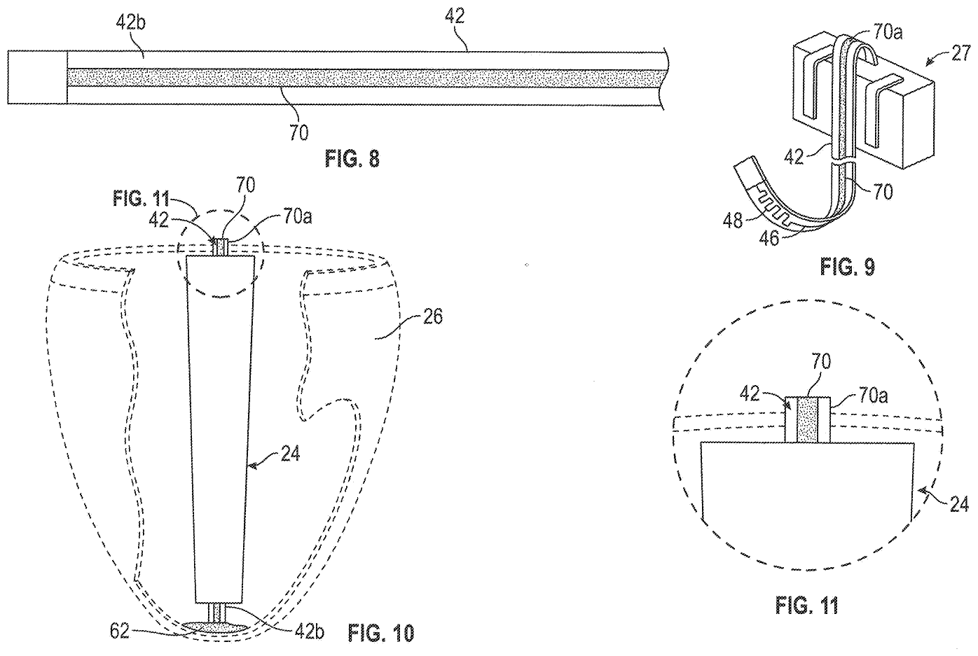

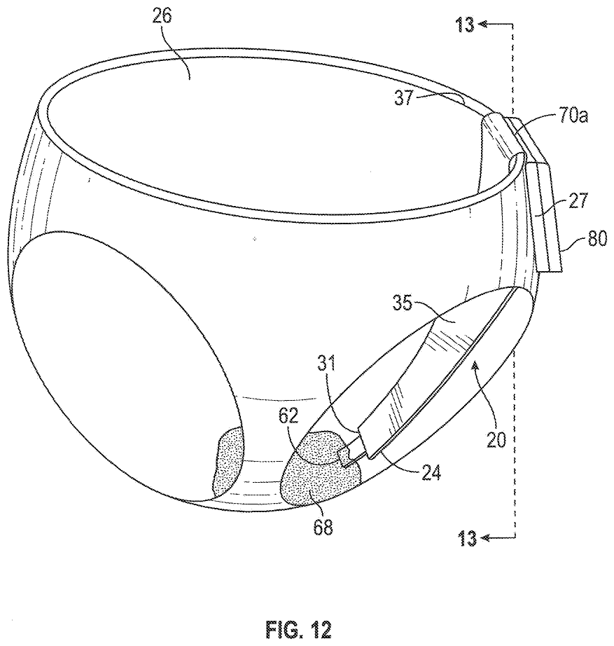

Disposable strip 22 (FIGS. 6 and 7) is preferably constructed from an elongated, generally rectangular strip of a highly efficient capillary liquid transfer wick material 42 designed to enhance absorption, retention and transfer of liquid by capillary attraction. A wick 42 having a length of about 7.75 inch, a width of about 0.5 inch and a thickness of about 0.063 inch may be used to practice the invention. A wick of this type may be purchased, for example, from Filtrona Richmond, Inc. of Richmond, Va. under the trademark name TRANSPAD®. Elongate wick strip 42 has a first, or outer side 42a and a second, or inner side 42b (FIGS. 6 and 8) and here comprises a substantially short first portion 23 which protrudes out of a first open end 31 (FIGS. 4 and 5) of diaper insert 24 and is intended for placement in the crotch area of a young child's or incontinent adult's diaper 26 (FIGS. 2 and 3) to allow rapid transfer of moisture (such as from urination) by capillary attraction to an adjacent substantially long second portion 25 (FIG. 6). In one example, first portion 23 may be approximately 0.75 inch long. Second portion 25 is preferably fully enclosed by the waterproof inner walls of diaper insert 24 to prevent loss of transferred urination to the diaper padding. Second portion 25 is also adapted on first side 42a (FIGS. 6 and 7) to conduct current in the presence of transferred moisture (urination) from the crotch area of the diaper. Wick strip 42 further comprises a third portion 21 (FIG. 6) disposed adjacent to second portion 25. Third portion 21 protrudes out of diaper insert 24 (FIG. 4) to allow for electrical coupling at an end 50 to an electrical circuit 52 (FIG. 6) which is part of a relatively compact portable and preferably re-usable alarm signal unit 27 (FIG. 1).

Alarm signal unit 27 contains a custom-made electronic circuit board (not shown) adapted to provide light and/or sound and/or vibratory alarm signals or the like to alert a caregiver that a young child or incontinent adult is in need of a diaper change. Alarm signal unit 27 may be adapted to send a radio signal to a receiver on a remote caregiver and is preferably removably coupled to diaper insert 24 by manually inserting alarm signal unit 27 in a pocket 29 formed in a tail portion 30 of diaper insert 24 (FIG. 1). In one example, portable alarm signal unit 27 may have the following approximate exterior dimensions: 1.5 inch length by 1.4 inch width by 0.2 inch thickness and may be powered by a small 3-volt battery (not shown).

Second side 42b of the wick strip 42 is preferably adhesively attached to the waterproof inner wall of diaper insert 24. Other means of securing wick 42 inside diaper insert 24 may be used, provided such other securing means agree with the intended purpose of the present invention.

Diaper insert 24 is preferably made from at least one layer of thin flexible material reinforced with a waterproof plastic backing layer on one side which, in one embodiment of the present invention, is cut, folded substantially in the middle such that the waterproof plastic backing remains on the inside to form the inner wall and then heat-sealed at the open edges on each of two opposing sides to form a relatively flat elongated pouch or envelope which is open at first end 31 (FIGS. 4 and 5). Pouch-like diaper insert 24 may be constructed from dental bib-type paper which is usually reinforced on one side with a thin plastic waterproof backing material. Dental-bib type paper may be purchased from medical/dental suppliers such as Henry Schein, Inc. of Melville, N.Y.

As generally illustrated in FIGS. 4-5, tail portion 30 of pouch-like diaper insert 24 is generally rectangular-shaped, closed at a second end 33 (since end 33 represents the line of folding the dental bib material to form diaper insert 24) and provided on a side 35 with a polygonal cutout 32 to allow third portion 21 of wick strip 42 to electrically couple with electrical circuit 52 (FIG. 7) of alarm signal unit 27. Cutout 32 also serves as the opening for pocket 29 (FIG. 1) of tail portion 30 of diaper insert 24. Tail portion 30 preferably folds over a diaper waistline 37 (FIGS. 2 and 3) to form pocket 29 on the outside of diaper 26 for inserting alarm signal unit 27.

A remaining portion 34 of diaper insert 24 is generally trapezoidal-shaped, open at first end 31 (FIGS. 4 and 5) to accommodate outwardly protruding substantially short first portion 23 of wick strip 42 and preferably provided at an opposite side 36 (FIG. 5) with a double-sided adhesive strip 38 which has a peelable backing 40 (FIG. 5). Backing 40 is peeled off by the caregiver to adhesively attach side 36 of diaper insert 24 to the inside of diaper 26 (FIGS. 2 and 3) such that first portion 23 of wick strip 42 is preferably disposed in the diaper crotch area (FIGS. 2 and 3) before the diaper can be used by a young child or an incontinent adult. Other means attaching diaper insert 24 to the inside of diaper 26 may be used such as sewing, laminating in place or the like, provided such other attachment means fall within the scope of the present invention.

In accordance with one embodiment of the present invention, side 42a of second and third portions of wick strip 42 is adapted to conduct electric current by screen-printing on it a pair of elongated, spaced apart and generally parallel conductive ink bands 44, 46 as generally depicted in FIGS. 6 and 7. Conductive ink bands 44, 46 are substantially identical, i.e. of the same width, length and overall configuration. The preferred spacing between conductive ink bands 44, 46 is about one half the width of a conductive ink band (44 or 46).

Conductive ink is formed by mixing carbon powder or silver powder with acrylic resin and solvent and then dispersing the same. Conductive ink may be applied to a variety of substrates by screen printing, dipping and syringe dispensing. Conductive ink suitable for practicing the invention may be purchased, for example, from Creative Materials Incorporated of Tyngsboro, Mass. The preferred thickness of each conductive band (44, 46) is about 0.002 inch-0.003 inch.

Conductive ink bands 44, 46 terminate at one end with a tortuous conductive ink pattern 48, one example of which is shown in FIGS. 6 and 7, designed to increase the moisture contact surface area. Tortuous conductive ink pattern 48 has a front end 64 (FIGS. 6 and 7) preferably disposed in close proximity to substantially short first portion 23. In one example, the distance moisture (urination) has to travel by capillary attraction between front end 64 and first portion 23 may be 0.75 inch. At the opposite end, conductive ink bands 44, 46 extend all the way to end 50 of third portion 21 of wick strip 42 (FIGS. 6 and 7) to couple electrically to leads 51, 53 of electrical circuit 52 (FIG. 7). A person skilled in the art would readily recognize that various other conductive ink patterns may be implemented in lieu of tortuous conductive ink pattern 48 to increase moisture contact surface area in close proximity to substantially short first portion 23 (which is placed in the crotch area of diaper 26), provided such other patterns fall within the scope of the present invention.

Electrical circuit 52 (FIG. 7) is shown as a simplified circuit including a power source or battery 54, an audio alarm device 56, a visual alarm (e.g., colored lights) device 58 and a manual switch 60. Circuit 52 may be disposed in a light-weight plastic housing (not shown) to form a portable alarm signal unit. A person skilled in the art would appreciate that other types of alarm devices may be connected to simplified circuit 52, e.g. a vibratory alarm device or the like.

Simplified circuit 52 is merely one example of an electrical circuit which may be used to practice the present invention. Other more complex circuits may be utilized depending on the needs of the caregiver providing such circuits do not deviate from the intended purpose of the present invention.

In accordance with the general principles of the present invention and assuming manual switch 60 in simplified electrical circuit 52 (FIG. 7) is closed, any amount of urine such as 68 (FIG. 2) in crotch area 66 of diaper 26 coming in contact with a tip 62 (FIG. 2) of first portion 23 of wick strip 42 is being quickly transferred up (via the highly efficient wick strip material 42) to tortuous conductive ink pattern 48 of second portion 25 by capillary attraction. Transferred moisture (from crotch area 66) coming in contact with tortuous conductive ink pattern 48 will short circuit the conductive ink bands 44, 46 (i.e. close circuit 52) triggering alarm devices 56, 58 so as to alert the caregiver that a diaper change is required. A person skilled in the art would readily appreciate that the distance moisture 68 travels to reach tortuous conductive ink pattern 48, which in the above example is about 1.5 inch, is substantially shorter than the distance moisture has to travel in the above-described prior art moisture detecting device which used a capillary strip placed in the diaper crotch area and integrated electrical wires (electrodes) disposed far away at the waistline of the diaper. In one example and in accordance with the general principles of the present invention, typical potential and current conducted between conductive ink bands 44, 46 may be about 3 volts and a few microamperes, respectively, i.e. novel moisture sensing apparatus 20 eliminates all of the above-mentioned prior art health safety concerns.

Screen-printing two spaced apart conductive ink bands directly on wick strip 42 instead of attaching or incorporating two plain electrical wires on wick strip 42 reduces the overall cost of the moisture sensing apparatus and provides additional advantages such as using lower potentials, conducting very low currents and the like. The screen-printed conductive ink bands are flexible electrical conductors with a larger overall surface current area than plain electrical wires and are also less likely to be damaged during use alleviating any potential safety concerns a caregiver or user may have.

An extremely important feature of the improved apparatus of the present invention resides in the provision on the second side 42b of wick strip 42 of a temperature sensor comprising a thermochromic material chosen from a group comprising leuco dyes. In one form of the invention, the thermochromic material comprises a strip 70 of thermochromic ink that is applied to the second side 40b of the wick along its entire length (see FIG. 8). Strip 70 preferably has a width of between 2 and 4 mm. As best seen by referring to FIGS. 3, 10 and 11 of the drawings, with this construction the portion 70a of strip 70 that is located proximate the interconnection of the wick 42 with the signal unit 27 is exposed so that the color change of the strip indicating an elevated temperature will be clearly visible to the caregiver.

The thermochromic ink used to form strip 70 comprises temperature-sensitive dyes (or inks) called leuco dyes. Leuco dyes are organic (carbon-based) chemicals that change color when heat energy makes their molecules shift back and forth between two subtly differently structures known as the leuco (colorless) and non-leuco (colored) forms which start off transparent (or have a particular color) and become visible (or change to a different color) as the temperature rises. Organic leuco dyes are available for temperature ranges between about −5° C. (23° F.) and 60° C. (140° F.), in wide range of colors. The color change usually happens in a 3° C. (5.4° F.) interval. While thermochromic inks are available from numerous sources, a thermochromic ink offered for sale by LCR Hallcrest LLC of Glenveiw, Ill. is suitable for forming strip 70.

In using the apparatus of the present invention in the manner illustrated in FIGS. 3 and 10 of the drawings, the thermochromic strip 70 will function to continuously monitor the temperature of the wearer of the diaper. In the event that the body temperature of the wearer exceeds normal body temperature of approximately 37° C., the strip will dramatically change color to immediately signal to the caregiver the existence of a higher than normal body temperature and fever.

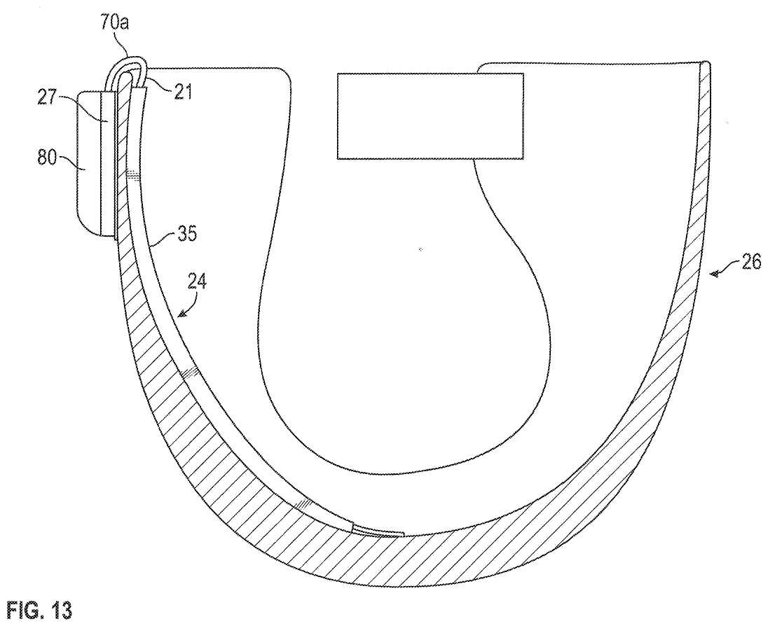

Referring now to FIGS. 12, 13, 14 and 15, still another embodiment of the present invention is there illustrated. This embodiment of the invention is similar in many respects to the embodiments of invention illustrated in FIGS. 1 through 11 of the drawings and described in the previous paragraphs. Accordingly, like numerals are used in FIGS. 12, 13, 14 and 15 to identify like components. As shown in FIG. 14 of the drawings, this latest form of the invention also uses an elongate wick strip 42. As before, a thermochromic material chosen from a group comprising leuco dyes is provided on the second side 42b of the wick strip. As illustrated in FIGS. 12 and 13 of the drawings, the second side 42b of the wick strip 42 is preferably adhesively attached to the waterproof inner wall of a diaper insert 24. As before, diaper insert 24 is preferably made from at least one layer of thin flexible material reinforced with a waterproof plastic backing layer on one side which, in one embodiment of the present invention, is cut, folded substantially in the middle such that the waterproof plastic backing remains on the inside to form the inner wall, and then heat-sealed at the open edges on each of two opposing sides to form a relatively flat elongated pouch or envelope which is open at first end 31. It is to be noted that, as previously discussed, the portion 70a of strip 70 that is located proximate the interconnection of the wick 42 with the signal unit 27 is exposed so that the color change of the strip indicating an elevated temperature is visible to the caregiver.

As previously discussed, in using the apparatus of the invention, the thermochromic strip 70 will function to continuously monitor the temperature of the wearer of the diaper. In the event that the body temperature of the wearer exceeds normal body temperature of approximately 37° C., the strip will dramatically change color to immediately signal the existence of a higher than normal body temperature and fever.

An extremely important feature of this latest embodiment of the invention resides in the provision of a color sensor 80, which, as illustrated in FIGS. 13 and 15, is mounted in close proximity to a temperature sensor 27. With the color sensor 80 in this position, when the wick strip 42 is inserted into the signal unit in the manner shown in FIG. 15, the conductive ink pattern 48 will face inwardly and the thermochromic strip 70 on the opposite side of the wick will face outwardly toward the color sensor. With this configuration, the sensor can readily sense a change in color of the thermochromic strip 70 and generate an appropriate signal 82 (FIG. 15). Color sensor 80 is readily commercially available from various sources, including the Banner Engineering Corporation of Plymouth, Minn., the TTI Company of Fort Worth, Tex., and RGB And Gesture Company having offices at Cupertino, Calif. Operably associated with the color sensor for receiving the signal 82 is a receiving and transmitting means 84. Receiving and transmitting means 84 can comprise either a blacktooth module or a Wi-Fi module, both of which are well understood by those skilled in the art and both of which can transmit data to applicable wireless platforms including multiple types of remote monitors or notification devices including for example computers 86, speakers 88 and cell phones 90 (FIG. 15).

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

I claim:

1. A patient care apparatus usable by a caregiver for monitoring the temperature of a patient, said apparatus including a notification device and comprising:
(a) an elongate strip of material disposed proximate the patient;
(b) an elongated band of thermochromic ink disposed on said elongate strip of material;
(c) a color sensor operably associated with said elongate strip of material for generating and transmitting a signal upon sensing a change in color of said thermochromic ink; and
(d) receiving and transmitting means operably associated with said color sensor for receiving said signal and for generating and transmitting a notifying signal to the notification device.

2. The apparatus as defined in claim 1 in which said receiving and transmitting means comprises a bluetooth module.

3. The apparatus as defined in claim 1 in which said receiving and transmitting means comprises a WiFi module.

4. The apparatus as defined in claim 1 in which said notification device comprises a computer.

5. The apparatus as defined in claim 1 in which said notification device comprises a cell phone.

6. The apparatus as defined in claim 1 in which said notification device comprises a speaker.

7. A patient care apparatus usable by a caregiver for providing care to a patient including monitoring the patient's temperature, said apparatus including a notification device and comprising:
(a) an elongate strip of material having a first side and a second side;
(b) an elongated band of thermochromic ink disposed on a selected side of said first side and second side of said elongate strip of material;
(c) a color sensor operably associated with said selected side of said elongate strip of material for generating and transmitting a signal upon sensing a change in color of said thermochromic ink; and
(d) receiving and transmitting means operably associated with said color sensor for receiving said signal and for transmitting a notifying signal.

8. The apparatus as defined in claim 7 in which said receiving and transmitting means comprises a blacktooth module.

9. The apparatus as defined in claim 7 in which said receiving and transmitting means comprises a WiFi module.

10. The apparatus as defined in claim 7 in which the notification device comprises a computer.

11. The apparatus as defined in claim 7 in which the notification device comprises a cell phone.

12. The apparatus as defined in claim 7 in which said notification device comprises a speaker.

13. The apparatus as defined in claim 7 in which said patient care apparatus is usable by a caregiver for sensing moisture and in which said elongate strip of material comprises:
(a) a capillary liquid transfer wick adapted to efficiently absorb and transfer moisture via capillary attraction from said first location to a second location;
(b) at least two spaced apart conductive ink bands disposed on said strip of wick material; and
(c) an elongated band of thermochromic ink disposed on said strip of wick material proximate said conductive ink bands.

14. The apparatus as defined in claim 13 in which said thermochromic ink comprises temperature-sensitive organic chemicals that change color when heat energy makes their molecules shift back and forth between two subtly differently structures.

15. The apparatus as defined in claim 13 in which said thermochromic ink comprises leuco dyes.

16. The apparatus as defined in claim 13 in which said thermochromic ink changes color at a temperature greater than 98.6 F.

17. The apparatus as defined in claim 13 further including a waterproof pouch for enclosing a portion of said wick, said waterproof pouch having a pocket.

18. The apparatus as defined in claim 17 further including an alarm unit disposed within said pocket of said waterproof pouch and operably associated with said receiving and transmitting means.

19. A patient care apparatus usable by a caregiver for providing care to a patient, said apparatus including a notification device and comprising:
(a) an elongate capillary liquid transfer wick disposed proximate the patient, said liquid transfer wick having a first side and a second side;
(b) an elongated band of thermochromic ink disposed on a selected side of said first side and second side of said liquid transfer wick;
(c) a color sensor operably associated with said selected side of said liquid transfer wick for generating and transmitting a signal upon sensing a change in color of said thermochromic ink;
(d) receiving and transmitting means operably associated with said color sensor for receiving said signal and for transmitting a notifying signal to the notification device;
(e) at least two spaced apart conductive ink bands disposed on said liquid transfer wick; and
(f) an elongated band of thermochromic ink disposed on said liquid transfer wick proximate said conductive ink bands.

20. The apparatus as defined in claim 19 in which said elongate capillary liquid transfer wick has first and second ends and a first portion at said first end and adapted to be disposed at said first location, a second portion disposed adjacent said first portion and a third portion disposed at said second end adjacent said second portion adapted to efficiently absorb and transfer moisture via capillary attraction from said first location to a second location disposed in close proximity to said first location
(a) at least two spaced apart conductive ink bands disposed on said strip of wick material; and
(b) an elongated band of thermochromic ink disposed on said strip of wick material proximate said conductive ink bands.
